(19) United States
(12) Patent Application Publication
(10) Pub. No.: US 2025/0231407 A1
(43) Pub. Date: Jul. 17, 2025

(54) RETINAL SCANNING DISPLAY DEVICE

(71) Applicant: BAE SYSTEMS PLC, London (GB)

(72) Inventor: Martyn Ingleton, Rochester Kent (GB)

(73) Assignee: BAE SYSTEMS PLC, London (GB)

(21) Appl. No.: 18/855,830

(22) PCT Filed: Apr. 12, 2023
(86) PCT No.: PCT/GB2023/050975

§ 371 Date: Oct. 10, 2024

(30) Foreign Application Priority Data
Apr. 13, 2022 (EP) 22275046.5
Apr. 13, 2022 (GB) 2205465.4

(51) Int. Cl. G02B 27/01; G02B 26/10; G02B 27/30; H04N 9/31

(52) U.S. Cl. CPC G02B 27/0172; G02B 26/101; G02B 26/103; H04N 9/3173; G02B 27/30

(57) ABSTRACT

A retina scanning display device (300) configured to provide an image to a retina of an eye (304) of a user, the device comprising: a light source (302) configured to generate intensity modulated light; an optic device (306) for transforming the intensity modulated light into a beam of collimated light; a scanning component (308) configured to receive the beam, scan the beam over at least two axes, and thereby output a series of scanned beams each having one of a plurality of an output angles; and a reflective combiner (320) arranged to reflect the scanned beams onto the eye over the range of output angles, whereupon the eye may focus the beam onto a portion of the retina in dependence on the output angle; wherein the beam has a predetermined diameter (d) greater than 3 mm.

### FIELD

The present invention relates to a retinal scanning display device particularly, but not exclusively for a visor on a helmet or a head mounted system.

### BACKGROUND

A retinal scanning display device allows light to be projected directly onto specific regions of a retina of a user, and allows a light beam to be scanned across the retina, to thereby display an image onto the retina.

Such retinal scanning display devices are, in general, configured to include a light source emitting a light beam, a scanner scanning the light beam emitted from the light source onto a retina and a light exit at which the light beam scanned by the scanner exits the retinal scanning display device into the eye. In most situations these display devices are head mounted. The retinal scanning display devices can be bulky and may give rise to discomfort if the user is wearing the device for a significant time

Typical retinal scanning display devices can also easily present misinformation to a user: if the eye moves whilst the light beam is being projected the image may not be true and include aberrations or worse. In addition, conditions may prevent a true image being generated. This could potentially lead to accidents, particularly where the user is piloting or driving a vehicle.

In addition, typical devices have a small pupil which means that if the eye moves during projection the image generated on the retina is moving and the eye is not fooled into seeing the image as intended. This has made retinal scanning display devices a non-preferred option in applications where precision is needed for example when piloting a vehicle such as an aircraft.

A need exists to find a simple retinal scanning display device which is lightweight and comfortable to wear. A further need is for a retinal scanning display device which presents a true image to the user and reduces the risks that the image contains misinformation.

### SUMMARY

According to a first aspect of the invention there is provided a retina scanning display device configured to provide an image to a retina of an eye of a user, the device comprising: a light source configured to generate intensity modulated light; an optic device for transforming the intensity modulated light into a beam of collimated light; a scanning component configured to receive the beam, scan the beam over at least two axes, and thereby output a series of scanned beams each having one of a plurality of an output angles; and a reflective combiner arranged to reflect the scanned beams onto the eye over the range of output angles, whereupon the eye may focus the beam onto a portion of the retina in dependence on the output angle; wherein the beam has a predetermined diameter greater than 3 mm.

The retina scanning display device according to claim 1 wherein the beam has a predetermined diameter greater than or equal to 10 mm.

The retina scanning display device may further comprise a controller operably connected to the light source and the scanning element and configured to coordinate the intensity modulation of the light source with the output angle of the scanned beam, so as to generate a predefined image at the retina.

The predetermined diameter may be between 3 mm and 20 mm, or may be between 9 mm and 11 mm.

The scanning component may comprise a tiltable mirror and a swivelling light pipe.

At the combiner, a percentage of reflectivity of the reflective combiner may vary based on the application and for example may be greater than 0% and less than or equal to 100%. As such the retina display device device may present a combination of a real-world view and virtual information in proportions based on the reflectivity.

The retina scanning display device may be configured to present virtual information comprising symbology associated with a real-world scene.

According to a second aspect, there is provided a helmet comprising the retina scanning display device of the first aspect. The helmet may comprise a visor.

According to a third aspect, there is provided a visor for use with the retina scanning display device according to the first aspect, wherein the visor is configured to provide the reflective combiner located between the scanning component and the retina.

According to a fourth aspect, there is provided a method to provide virtual information directly to a retina of an eye of a user the method comprising: generating intensity modulated light at a light source; transforming the intensity modulated light into a beam of collimated light with a diameter greater than 3 mm; scanning the modulated beam over at least two directions to produce a scanned beam; and directing the scanned beam to the retina via a reflective combiner such that the beam raster scans the retina to project an image directly onto the retina.

### BRIEF DESCRIPTION OF THE FIGURES

Embodiments of the invention will now be described by way of example only with reference to the figures, in which:

FIG. 1 shows a schematic view of a helmet according to an aspect of the present invention;

FIG. 2 shows a representation of how a retinal scanning display device projects light to an eye;

FIG. 3a shows an optical diagram of a retinal scanning display device according to an aspect of the present invention at a first point in time;

FIG. 3b shows the retinal scanning display device of FIG. 3a at a second point in time;

FIG. 4 is a flow diagram illustrating how symbology is presented to an eye of the user.

### DETAILED DESCRIPTION

The present invention relates to a retinal scanning display device for use in a head mounted device. The retinal scanning display device is capable of scanning a light beam or a series of light beams across the eye such that light is sequentially addressed, as focussed spots, to specific regions of the retina. The intensity of the light beam can vary during the scan. Further, the scan occurs at sufficient speed such that, given the persistence of the retina, the user views an integrated image. In this way a user can be provided with at least one of a real image, a virtual image and an augmented image having real and virtual components. The retinal scanning display device is configured to scan a beam of light across the eye to form the image on the eye's retina. The beam is modulated and/or collimated as an example and as is described below.

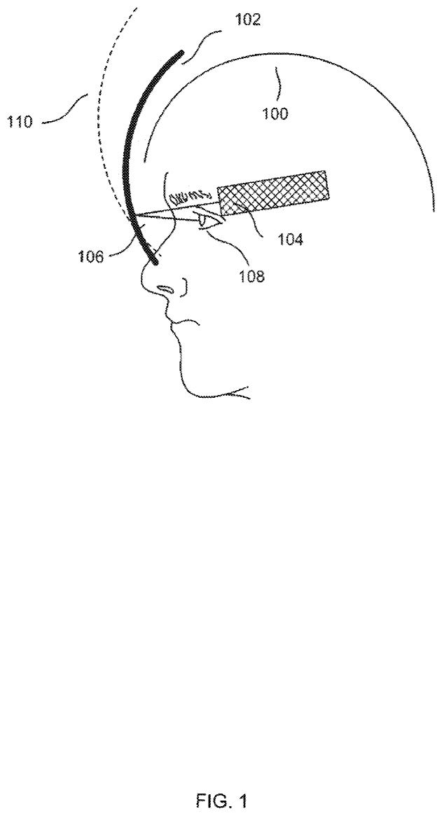

FIG. 1 shows a helmet 100 equipped with a visor 102 and including side mounted optics 104 which will be described in greater detail below. The visor forms part of the optics for projecting light 106 from the side mounted optics 104 to an eye 108 of a user. FIG. 1 also shows a nominal position of an existing visor position 110 for demonstrating that the present invention enables a more compact overall helmet due to the nature of the optics and that the visor 102 conforms more closely to the standard shape of the helmet 100.

The side mounted optics 104 also provide certain advantages in that the there is less obstruction to the view of the user and the positioning is aligned more closely to the centre of gravity of the helmet 100 giving rise to less physical strain for the user.

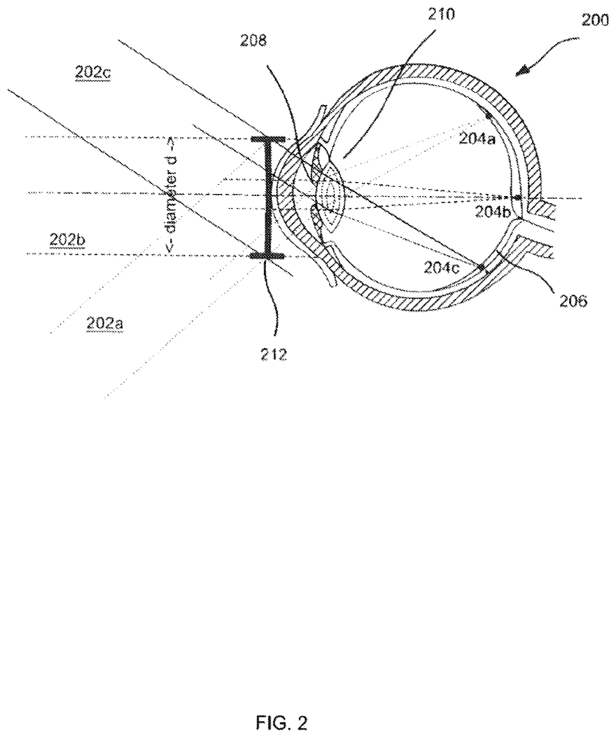

Referring to FIG. 2, when the eye 200 is illuminated with a beam 202b such as a collimated beam, along the axis defined by the eye, a focused spot 204b is formed at the centre of the retina 206. The figure shows spots 204a, 204b, 204c, each associated with a respective collimated beam 202a, 202b 202c, and being formed at different regions of the retina 206 depending on the field angle which the beam submits to the eye.

Each collimated beam enters the eye through the eye pupil 208 and is focused onto the retina by eye lens 210. (The eye pupil 208 is an aperture at the eye defined by iris 208). Light is scanned across the eye pupil 208 by inputting a collimated beam or a series of collimated beams having a range of angles and the brightness from beam to beam is modulated such that the brain will recognize the aggregated spots of light as a real image. The modulation is an adjustment in instantaneous brightness between 0 (zero) to the full display brightness. Brightness may be selected to cause persistence in the retina to generate the illusion of an image once the raster scan is completed. The angle of the scan directly relates to the display FOV. The smallest angle of scan to generate a pseudo image is the smallest angle to which the eye can perceive an image, for practical purposes the would be about 0.05 degrees. The scanning comprises raster scanning the collimated beam over the retina and provides an effective pupil 212 (which may alternatively be referred to as an eyebox) into the eye.

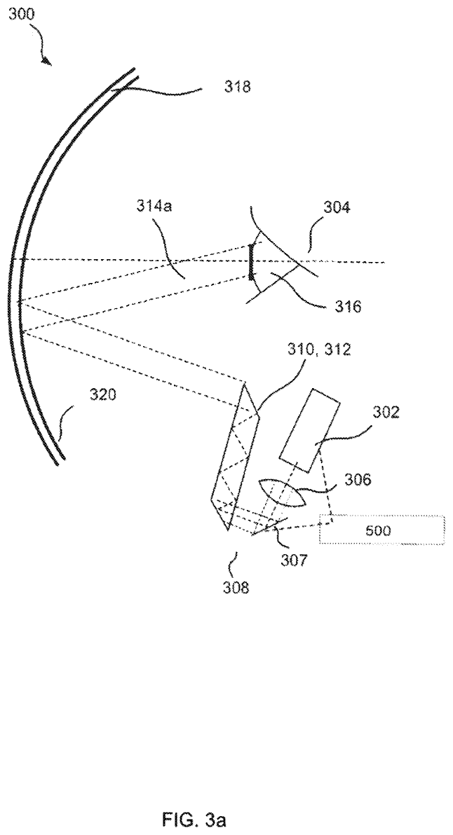

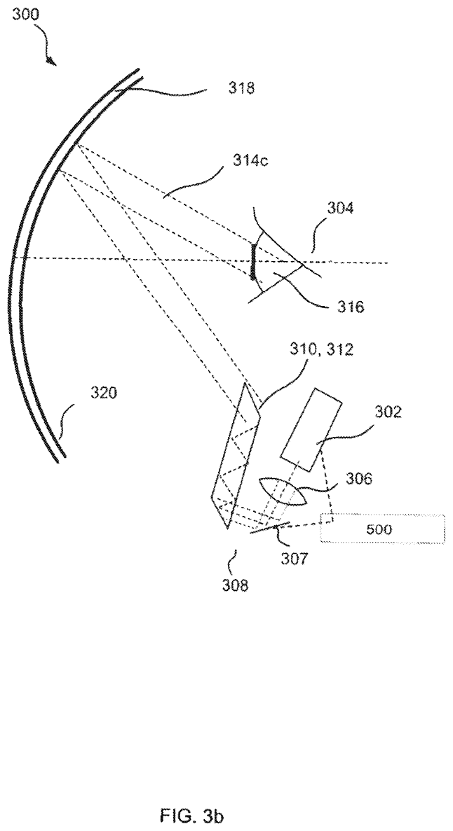

The beams from different field angles comprise colour beams of bright light from Light Emitting Diodes (LEDs), lasers or any other appropriate source. The colours could be all the same or different. The beams are then conditioned through a lens system and directed to the visor via a scanning element to generate the display field angles as illustrated with reference to FIG. 3.

FIGS. 3a and 3b show a schematic diagram of a retinal scanning display device 300.

The device 300 includes a modulated light source 302 which is projected onto the eye 304 of a user via optics.

The optics include a correction optic device 306 which comprises one or more lenses or any other appropriate optical element or elements. Such an optic device 306 is configured to output a collimated light beam with a wider diameter than the light input from the light source 302.

In addition, the optics comprise a scanning optical component 308 which comprises: an elevation scanning element 307; and an azimuth scanning element 312.

The elevation scanning element 307 comprises a tilting mirror or other suitable scanning display device.

The azimuth scanning element 312 comprises a swivelling light pipe 310 (i.e. one that is configured to swivel). The swivelling light pipe may oscillate back and forth between +30 degree and −30 degree angular displacements from a central alignment position.

The scanning is over two axes in a raster (e.g. line by line) scanning process and controlled by a controller 500. The controller 500 is operably connected to the scanning component 308 and the light source 202.

The controller coordinates the modulation of the light (e.g. the intensity) at the light source 302 with the position on the retina where the light will be focussed. In general the controller will infer the position on the retina where the light will be focussed from the instantaneous state of the scanning component 308. Thus light is presented to the eye at the desired angle and intensity and point in time, so as to ensure the required image is viewed by the user. The controller adapts the presentation of information so that the combination of the real world view and any virtual information are presented at a focal point that avoids unnecessary eye strain. This will generally mean that the real and virtual information appear to come from the same position. The modulated light source 302, the correction optics 306, the scanning optical components 308, 310 and 312 are located in the side mounted optics 104 shown in FIG. 1 or any other optics module for use with the helmet or head mounted device.

The optics are made from lightweight materials to avoid any strain on the user. The materials include aluminium, plastics, carbon composites and rubbers.

The light output from the scanning element 308, the scanned beams, are in the form of collimated light bundles (e.g. 314a, 314b, and 314c) having a diameter d. The diameter d is selected to be of the order of 10 mm which is an approximate size of but greater than the pupil 316 of the eye 304. The diameter is variable and can be greater or smaller than 10 mm and be with a range of 3 mm to 20 mm, or with a range of 9 mm to 11 mm, or within a range of 10 mm to 15 mm. The beam with diameter d is directed to a visor 318 of the helmet. The diameter d is selected so as to be bigger than the eye pupil which in daytime is about 3 mm. This will give tolerance to allow the eye to be able to move in its pupil plane without losing the display source (i.e. without travelling out of alignment with the eyebox). In practice the eye can move up to 5 mm in the pupil plane from the perfect alignment position before the display starts to cut off and eventually disappear when the display pupil 212 is outside of the pupil of the eye. The larger the eye pupil the larger the challenge to provide an appropriate optical schema.

This display pupil size 212 is large compared with previous retinal scanning display devices. The image is focussed on the retina over a number of eye rods. By configuring the beams to achieve this the movement of the eye has less impact as the projected beam illuminates a consistent portion of the retina even if the eye moves.

The visor 318 includes a partially mirrored inner surface 320 which directs the modulated collimated beams 314 towards the pupil 316 and in conjunction with the optics, is configured to scan the beam of modulated collimated light across the eye to form the image on the eye's retina (not shown in FIG. 3). The partial mirror allows external images to also be directed to the eye of the user. This could be a real environment or scene viewable by the user based on location and position. The percentage of reflectivity of surface 320 will depend on the application. The lower the percentage the dimmer any the virtual information will be, and the outside world will appear brighter A typical reflectivity would be about 50% where equal weight is given to the real and virtual information or images. If there is no need to see the outside world then 100% reflection could be used.

The visor could be replaced with another reflective combiner other than the visor in applications where there is no visor. An example may be goggles having at least a portion of the internal surface including a partial mirrored surface for directing the collimated beam to the retina. The partially mirrored inner surface is configured to reflect a percentage for the incident collimated beam based on the application.

External light from a scene or a cockpit can also enter the eye through the visor or reflective combiner so the user can view a real image and the collimated scanned modulated beams.

The real image shows a real world representation of the scene or environment in which the user is located. In the case of a pilot in a vehicle this includes the view from outside the vehicle and any controls within the cockpit or driving position of the user.

The collimated scanned modulated beams display a virtual image of information that is germane to the activity of the user. The virtual information comprises symbology, vehicular and scene information. The vehicular and scene information or data includes position, location, speed, temperature, radar or other sensor data which is from the scene or relating to the vehicle. It will be appreciated that the above information or data can be of any type coming from any appropriate sensor.

The retinal scanning display device could be used in different environments and is not limited for use on a helmet or head worn device. In the situation of a head worn device this could be a helmet and visor as shown in FIG. 1, or could be any other type of head worn device. The reflective combiner can form part of the retinal scanning display device or can be part of a helmet or goggles that has been configured to act as the reflective combiner for the retinal scanning display device.

In the case of head worn devices the side mounted optics could be located in different locations on the helmet although the optimal location is side mounted as shown, to reduce possible strain to the user.

The materials are just examples and other appropriate materials could be used.

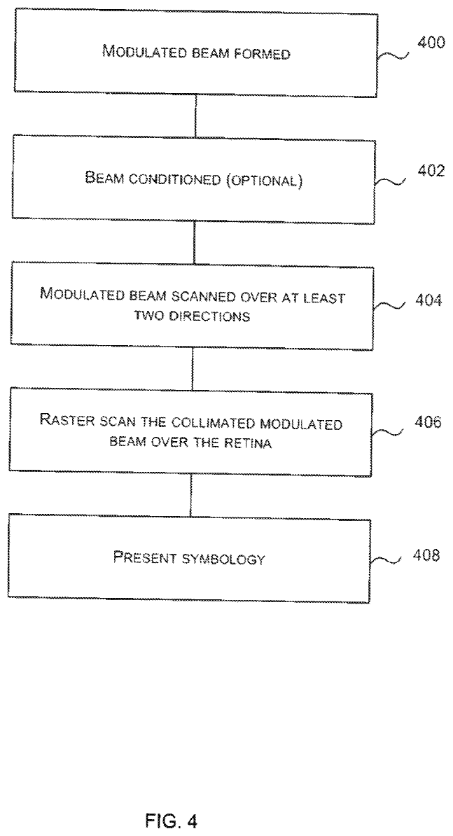

FIG. 4 shows a flow diagram illustrating how a retina scanning display device operates. A beam such as a modulated beam as an example, is formed 400. Optionally the beam is conditioned 402. The beam is scanned 404 over at least two directions to produce a collimated modulated beam having a predetermined diameter. Direct 406 the collimated modulated beam to the retina via a reflective combiner such that the beam raster scans the retina to project an image directly onto the retina. The raster scan presents symbology which can contain aid to help the user to drive the vehicle and/or sensor data 408 relating to the activities of a user or relating to the user's circumstances.

Raster scanning may be a line by line scan, where an image is built up as an array of pixels, or alternatively the scan may be a vector scan where the path of the scan traces out the image.

Symbology can include at least one or more of the following:
Signs and symbols
Data from sensors
Processed data from sensors
Combinations of sensor data
Military symbology
Vehicle related symbology
Scene related symbology.
Location and positioning symbology
Map symbology
Speed and velocity symbology

It should be noted that the light imaged in the present invention in the real or virtual information can be outside optical frequencies and include infra-red, ultra-violet and any other appropriate frequencies.

It will be appreciated the invention is described in as described above, but many variations and alternatives will be evident to the skilled person.

1. A retina scanning display device configured to provide an image to a retina of an eye of a user, the device comprising:
a light source configured to generate intensity modulated light;
an optic device for transforming the intensity modulated light into a beam of collimated light;
a scanning component configured to receive the beam, scan the beam over at least two axes, and thereby output a series of scanned beams each having one of a plurality of an output angles; and
a reflective combiner arranged to reflect the scanned beams onto the eye over the range of output angles, whereupon the eye may focus the beam onto a portion of the retina in dependence on the output angle;
wherein the beam has a predetermined diameter (d) greater than 3 mm.

2. The retina scanning display device according to claim 1, wherein the predetermined diameter (d) is greater than or equal to 10 mm.

3. The retina scanning display device according to claim 1, further comprising a controller operably connected to the light source and the scanning element and configured to coordinate the intensity modulation of the light source with the output angle of the scanned beam, so as to generate a predefined image at the retina.

4. The retina scanning display device according to claim 1, wherein the predetermined diameter (d) is between 3 mm and 20 mm.

5. The retina scanning display device according to claim 1, wherein the predetermined diameter (d) is between 9 mm and 11 mm.

6. The retina scanning display device according to claim 1, wherein the scanning component comprises a tiltable mirror and a swivelling light pipe.

7. The retina scanning display device according to claim 1, wherein a percentage of reflectivity of the reflective combiner varies based on the application and is greater than 0% and less than or equal to 100%.

8. The retina scanning display device according to claim 7, further configured to present a combination of a real-world view and virtual information via the scanning display device in proportions based on the reflectivity.

9. The retina scanning display device according to claim 1, wherein the scanning display device is configured to present virtual information comprising symbology associated with a real-world scene.

10. A helmet comprising the retina scanning display device according to claim 1.

11. The helmet of claim 10, wherein the helmet further comprises a visor.

12. A visor for use with the retina scanning display device according to claim 1, wherein the visor is configured to provide the reflective combiner located between the scanning component and the retina.

13. A method to provide virtual information directly to a retina of an eye of a user, the method comprising:
generating intensity modulated light at a light source;
transforming the intensity modulated light into a beam of collimated light with a diameter greater than 3 mm;
scanning the modulated beam over at least two directions to produce a scanned beam; and
directing the scanned beam to the retina via a reflective combiner such that the beam raster scans the retina to project an image directly onto the retina.

14. The method according to claim 13, further comprising: coordinating the intensity modulation of the light source with an output angle of the scanned modulated beam, so as to generate a predefined image at the retina.

15. The retina scanning display device according to claim 2, further comprising a controller operably connected to the light source and the scanning element and configured to coordinate the intensity modulation of the light source with the output angle of the scanned beam, so as to generate a predefined image at the retina.

16. A retina scanning display device configured to provide an image to a retina of an eye of a user, the device comprising:
a light source configured to generate intensity modulated light;
an optic device for transforming the intensity modulated light into a beam of collimated light;
a scanning component including a tiltable mirror and configured to receive the beam, scan the beam over at least two axes, and thereby output a series of scanned beams each having one of a plurality of an output angles; and
a reflective combiner arranged to reflect the scanned beams onto the eye over the range of output angles, whereupon the eye may focus the beam onto a portion of the retina in dependence on the output angle, wherein a percentage of reflectivity of the reflective combiner is greater than 0% and less than or equal to 100%;
wherein the beam has a predetermined diameter (d) greater than 3 mm.

17. The retina scanning display device according to claim 16, wherein the predetermined diameter (d) is greater than or equal to 10 mm.

18. The retina scanning display device according to claim 16, further comprising a controller operably connected to the light source and the scanning element and configured to coordinate the intensity modulation of the light source with the output angle of the scanned beam, so as to generate a predefined image at the retina.

19. The retina scanning display device according to claim 16, wherein the predetermined diameter (d) is between 3 mm and 20 mm.

20. The retina scanning display device according to claim 16, wherein the predetermined diameter (d) is between 9 mm and 11 mm.
